(19) United States
(12) Patent Application Publication
(10) Pub. No.: US 2023/0233544 A1
(43) Pub. Date: Jul. 27, 2023

(54) T-TYPE CALCIUM CHANNEL ENHANCER FOR TREATING TAF1 ASSOCIATED NEUROLOGICAL DEFECTS

(71) Applicant: ARIZONA BOARD OF REGENTS ON BEHALF OF THE UNIVERSITY OF ARIZONA, Tucson, AZ (US)

(72) Inventors: Mark A. Nelson, Tucson, AZ (US); Rajesh Khanna, Tucson, AZ (US); Janakiraman Udaiyappan, Tucson, AZ (US); Dhanalakshmi Shankara Raman, Tucson, AZ (US)

(21) Appl. No.: 18/001,519

(22) PCT Filed: Jun. 11, 2021
(86) PCT No.: PCT/US21/37085

§ 371 Date: Dec. 12, 2022

Related U.S. Application Data
(60) Provisional application No. 63/038,346, filed on Jun. 12, 2020.

(51) Int. Cl. A61K 31/454; A61P 25/14

(52) U.S. Cl. CPC A61K 31/454; A61P 25/14

(57) ABSTRACT

Dysregulation of TAF1 function by various mechanisms can lead to disease in the central nervous system, such as the TAF1 Intellectual Disability (ID) Syndrome which currently has no therapeutic treatments. The present invention indicates that a novel T-type calcium channel enhancer, such as SAK3, has disease-modifying effects in animal models of TAF1 editing. In addition, the present invention provides insights into the molecular mechanism by which SAK3 exerts in pharmacologic effects. Moreover, the present findings imply that the T-Type voltage-gated calcium channels are novel molecular targets to develop therapeutics to treat TAF1 ID syndrome and that SAK3 is an attractive drug candidate to treat TAF1 associated neurologic disorders.

### CROSS-REFERENCES TO RELATED APPLICATIONS

This application claims benefit of U.S. Provisional Application No. 63/038,346 filed Jun. 12, 2020, the specifications of which are incorporated herein in their entirety by reference.

### FIELD OF THE INVENTION

The present invention relates to a method to treating and/or preventing motor deficit associated with TAF ID Syndrome, more particularly to a method that stimulates T-type Ca2+ channels through the use of a therapeutic amount of SAK3.

### BACKGROUND OF THE INVENTION

The transcription factor IID (TFIID) complex is an assembly of the TATA-box binding protein (TBP) and 12-14 TBP-associated factors (TAFs), participating in the preinitiation complex that initiates transcription of RNA polymerase II transcription-dependent genes. TAF1 is the largest TAF unit of the TFIID complex and plays a vital role in the preinitiation complex by facilitating binding to promoter regions. The TAF1 gene (GRCh37/hg19, chrX: 70586114-70685855, NM_004606.4 in humans) includes 39 exons and yields more than 20 coding and non-coding transcripts expressed in various tissues, including the central nervous system. Variants of TAF1 have been linked to neurodevelopmental disorders. TAF1 Intellectual Disability (ID) syndrome (also known as mental retardation, X-linked, syndromic-33 disease, MRXS33, OMIM: 300966) occurs mainly in males, leading to abnormalities in global developmental (motor, cognitive, and speech), hypotonia, gait abnormalities, and cerebellar hypoplasia. Only missense variants have been reported; the lack of hemi- and homozygous loss-of-function variants in the protein-coding part of the canonical TAF1 isoform in human population databases suggests that a complete loss of TAF1 may be embryonic-lethal. A non-coding 2.6 kb insertion of a SINE-VNTR-Alu (SVA)-type retrotransposon in intron 328 of TAF1 causes the neurological disorder X-linked dystonia-parkinsonism (XDP; OMIM: 313650). XDP is a progressive neurodegenerative disorder characterized by involuntary movements (dystonia), most often developing in adult life in combination with Parkinsonism. Taken together, these observations suggest that dysregulation of TAF1 function by different mechanisms can lead to disease in the central nervous system.

Exactly how variants in TAF1 give rise to these neurological deficits remains unclear. TAF1 serves as a scaffold for the assembly of the transcription factor TFIID complex. Furthermore, how the many functions of the TFIID complex are linked to the development of TAF1 ID syndrome is unknown. This has led to a lack of therapeutic strategies to treat TAF1 ID syndrome. Hence, there exists a need for treatments of TAF1 ID syndrome.

### BRIEF SUMMARY OF THE INVENTION

It is an objective of the present invention to provide a method of treating, preventing, and/or improving motor deficits associated with TAF1 Intellectual Disability Syndrome (TAF1 ID syndrome), as specified in the independent claims. Embodiments of the invention are given in the dependent claims. Embodiments of the present invention can be freely combined with each other if they are not mutually exclusive.

Recently an animal model of TAF1 ID syndrome was created using CRISPR/Cas9 gene editing. This animal model provided a powerful new tool to study the neuronal dysfunction in conditions associated with TAF1 abnormalities. It was found that in neonatal rat pups, removal of TAF1 by CRISPR/Cas9 editing, results in defects in neonatal motor functions. Furthermore, the motor deficits were associated with loss of Purkinje cells (PC) in the cerebellum; the remaining Purkinje cells displaying abnormal firing frequencies. In addition, the motor defects persisted in TAF1-edited juvenile pups and were associated with morphological abnormalities within the cerebellum and cerebral cortex. TAF1 regulates the expression of the CaV3.1 T-type Ca2+ channel in vitro and in vivo. Furthermore, the CaV3.1 T-type channel accounts for the reduction in spontaneous excitatory postsynaptic currents in TAF1-edited animals. These observations suggest that impaired Cav3.1 T-type channel activity plays a functional role in the pathogenesis of TAF1 ID syndrome.

T-type calcium channels are low voltage-activated calcium channels that transiently open to evoke tiny Ca2+ currents. T-type calcium channels play a crucial role in regulating intracellular calcium homeostasis and maintaining cellular function. Cav3.1 T-type channels are abundant at the cerebellar synapse between parallel fibers and Purkinje cells where they contribute to presynaptic depolarization.

The present invention investigated the effects of SAK3 on the behavioral and pathophysiological defects associated with TAF-1 gene editing in the TAF1 ID syndrome animal model. In addition, the molecular mechanisms of the underlying SAK3 effects were also evaluated. The present invention provides compelling evidence that T-type Ca2+ channel stimulation can have disease-modifying effects in TAF-1 edited animal models.

The present invention features a method of treating, preventing, and/or improving motor deficits associated with TAF1 Intellectual Disability Syndrome (TAF1 ID syndrome) in a subject in need of such treatment. In some embodiments, the method comprises administering a therapeutic amount of SAK3. The method may be capable of treating motor deficits associated with TAF1 ID Syndrome such that clinical improvements are observed. The present invention also features a method of restoring protein levels and regenerating cells back to normal in vivo by administering SAK3.

One of the unique and inventive technical features of the present invention is the administration of SAK3 as a therapeutic treatment for motor deficits associated with TAF1 ID Syndrome. Without wishing to limit the invention to any theory or mechanism, it is believed that the technical feature of the present invention advantageously provides for disease-modifying effects that allow for the regeneration of Purkinje cells as well as astrocytes. Additionally, the present invention also allows for restoring the protein levels of FOXP2 and BDNF. None of the presently known prior references or work has the unique inventive technical feature of the present invention.

Furthermore, the prior references teach away from the present invention. For example, prior references have found inhibition of the T-type channel activity to be efficacious for neurological diseases. However, the present invention has found that enhancement of T-type channel activity was beneficial for neurological diseases. Additionally, the present invention discovered that SAK3 restored the levels of FOXP2, a gene responsible for speech in humans, back to normal levels in TAF1-edited animals.

Furthermore, the inventive technical features of the present invention contributed to a surprising result. For example, SAK3 was found to be neuroprotective for the cerebellum and the cerebral cortex. The administration of SAK3 helped to repopulate Purkinje cells and astrocytes after the animal displayed motor deficits. This was surprising because it is not often thought that brain cells can be regenerated after they are lost.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1A shows the experimental design for electrophysiology, behavioral, histopathological, and molecular studies.

FIGS. 1B, 1C, and 1D show that the TAF1-edited animals showed behavior deficits compared to naïve and gRNA-control group animals when looking at beam crossing time (FIG. 1B), the number of foot slip errors (FIG. 1C), and the number of groomings (FIG. 1D). SAK3 administration to the TAF1 edited animals showed improved the TAF1 edited behavioral abnormalities. Data are shown as mean±S.E.M., n=24 per experimental condition. *p<0.05 versus; naive, #p<0.05 versus gRNA-control (ANOVA followed by Tukey's test). The experiments were conducted in an investigator blinded manner.

FIG. 2A shows the morphology of the cerebral cortex was evaluated Nissl Staining. The Nissl staining showed shrunken neurons with vacuolated intercellular spaces and many unstained regions of the cortical neurons after TAF1 gene editing. SAK3 administration to the TAF1 edited animals improved the morphology of the cortical neurons.

FIG. 2B shows the enumeration of the cells in each treatment group. Note decreased cell viability in TAF-1 edited animals compared to the control groups and animals treated with SAK3.

FIGS. 2C and 2D show the expression of calbindin was decreased in TAF1-edited animals as compared to naïve and CRISPR-control groups. SAK3 administration to the TAF1 edited animals shows an increased number of Calbindin positive Purkinje cells. Summary of the number of Purkinje cells per linear density (FIG. 2B) in each of the experimental conditions. Data are shown as mean±S.E.M., n=12 fields per animal, four animals per experimental condition. *p<0.05 versus; naive, #p<0.05 versus gRNA-control (ANOVA followed by Tukey's test). Scale bars: 200 μm. The experiments were conducted in a blinded fashion.

FIGS. 2E and 2F show the expression of GFAP was decreased in TAF1-edited animals as compared to naïve and CRISPR-control groups. SAK3 administration to the TAF1 edited animals shows an increased number of GFAP positive cells. Summary of the number of GFAP positive cells (FIG. 2D) in each of the experimental conditions. Data are shown as mean±S.E.M., n=12 fields per animal, 4 animals per experimental condition. *p<0.05 versus; naive, #p<0.05 versus gRNA-control (ANOVA followed by Tukey's test). Scale bars: 200 μm. The experiments were conducted in a blinded fashion.

FIGS. 2G and 2H show the expression of IBA-1 (microglia marker) was increased in TAF1-edited animals as compared to all other experimental groups. SAK3 administration to the TAF1 edited animals showed a decrease in the number of IBA-1 -positive cells as compared to the TAF1 edited group. Summary of the number of IBA-1 positive cells (FIG. 2F) in each of the experimental conditions. Data are shown as mean±S.E.M., n=12 fields per animal, four animals per experimental condition. *p<0.05 versus; naive, #p<0.05 versus gRNA-control (ANOVA followed by Tukey's test). Scale bars: 200 pm. The experiments were conducted in a blinded fashion.

FIG. 3A shows a photomicrograph of cerebellar slice preparation with a progressive zoom with the rightmost panel showing the positioning of the recording electrode to this region.

FIG. 3B shows representative recording traces of cells from the indicated groups.

FIG. 3C and 3D show the summary of amplitudes (FIG. 3C) and frequencies (FIG. 3D) of sEPSCs for the indicated groups are shown. Data are shown as mean±S.E.M., n=12 Purkinje cells from at least 2 animals per experimental condition. *p<0.05 versus; gRNA-control, &p<0.05 versus gRNA-TAF1 (ANOVA followed by Tukey's test). The experiments were conducted in an investigator-blinded manner.

FIG. 4A shows photomicrographs from gRNA-TAF1 edited animals, and gRNA-TAF1 edited animals treated with SAK3. The data from the control groups can be found in FIG. 6. Note SAK3 reduced the number of TUNEL positive cells in gRNA-TAF1 edited animals.

FIG. 4B shows the summary of the number of TUNEL positive cells in each of the experimental conditions. Data are shown as mean±S.E.M., n=12 fields per animal, 4 animals per experimental condition. *p<0.05 versus; naive and gRNA-TAF1=SAK3 group (ANOVA followed by Tukey's test). Scale bars: 200 μm. The experiments were conducted in an investigator-blinded manner.

FIGS. 5A and 5B shows a representative western analysis is shown from cerebral cortex samples from animals from each of the experimental conditions. Note increased cleaved caspase 3 in gRNA-TAF1 edited animals and diminished cleaved caspase 3 levels in gRNA-TAF1 edited animals and diminished cleaved caspase 3 levels in gRNA-TAF1 SAK3 treated animals (FIG. 5A). Quantification of Western analysis from independent experiments. Data shown are mean±SEM, n=6 animals per each experimental condition. *p<0.05 versus gRNA-control water and RNA-control SAK3 group; #p<0.05 versus gRNA-TAF1 water group (ANOVA followed by Tukey's test) (FIG. 5B)

FIGS. 5C and 5D show an increase of BAX levels in gRNA-TAF1 edited animals, whereas SAK3 reduced BAX levels to control levels on a representative western blot (FIG. 5C). Quantification of Western analysis from independent experiments. Data shown are mean±SEM, n=6 animals per each experimental condition. *p<0.05 versus gRNA-control water and RNA-control SAK3 group; #p<0.05 versus gRNA-TAF1 water group (ANOVA followed by Tukey's test) (FIG. 5D)

FIGS. 5E and 5F show a decrease of Bcl-2 levels in gRNA-TAF1 edited animals, whereas SAK3 increases Bcl-2 expression to control levels on a representative western blot (FIG. 5E). Quantification of Western analysis from independent experiments. Data shown are mean±SEM, n=6 animals per each experimental condition. *p<0.05 versus gRNA-control water and RNA-control SAK3 group; #p<0.05 versus gRNA-TAF1 water group (ANOVA followed by Tukey's test) (FIG. 5F).

FIG. 6 shows the effects of SAK3 on the cerebral cortex of Naïve and gRNA-control animals. Apoptosis was assessed in cerebral cortex samples using a TUNEL assay. Shown are TUNEL assay photomicrographs from Naive and gRNA-control animals treated with SAK3. Note water and SAK3 treatment to the naïve and gRNA-control animals had no significant change in TUNEL positive cells. Scale bars: 200 μm. The experiments were conducted in an investigator-blinded manner.

FIG. 7A shows the expression of BDNF was decreased in TAF1-edited animals (Av) as compared to all other experimental groups (Ai-iv). SAK3 administration to the TAF1 edited animals shows an increased number of BDNF-positive cells as compared to the TAF1 edited group (Avi & v).

FIG. 7B shows the summary of BDNF expression. Data are shown as mean±S.E.M., n=12 fields per animal, four animals per experimental condition. Scale bars: 200 μm.

FIG. 7C shows representative Western analysis of BDNF is shown from cerebral cortex samples from animals from each of the experimental conditions. Note decreased BDNF levels in gRNA-TAF1 edited animals, whereas SAK3 increases BDNF expression to control levels.

FIG. 7D shows the quantification of Western analysis from three independent experiments. Data shown are mean±SEM, n=6 animals per each experimental condition. *p<0.05 versus; naïve and gRNA-TAF1 =SAK3 group (ANOVA followed by Tukey's test). The experiments were conducted in an investigator-blinded manner.

FIG. 8A shows the expression of p-AKT was decreased in TAF1-edited animals (Av) as compared to all other experimental groups (Ai-iv). SAK3 administration to the TAF1 edited animals shows increased expression of p-AKT as compared to the TAF1 edited group (Avi & v).

FIG. 8B shows the Summary of p-AKT expression. Data are shown as mean±S.E.M., n=12 fields per animal, four animals per experimental condition. Scale bars: 200 μm.

FIG. 8C shows representative western analysis of p-GSK36 is shown from cerebral cortex samples from animals from each of the experimental conditions. Note decreased p-GSK3β levels in gRNA-TAF1 edited animals, whereas SAK3 increases p-GSK3β expression to control levels.

FIG. 8D shows the quantification of western analysis from three independent experiments. Data shown are mean±SEM, n=6 animals per each experimental condition. *p<0.05 versus; naïve and gRNA-TAF1=SAK3 group (ANOVA followed by Tukey's test). The experiments were conducted in an investigator-blinded manner.

FIGS. 9A and 9B show the effects of SAK3 on GSK36 in the cerebellum. FIG. 9A shows a representative Western analysis is shown from cerebellum samples from each of the experimental conditions. Note decreased p-GSK3β in gRNA-TAF1 edited animals, which is rescued by SAK3 treatment. FIG. 9B shows the quantification of p-GSK3β expression on all the experimental conditions. Data shown are mean±SEM, n=4 animals per each experimental condition.

FIG. 10A shows TAF-1 editing decreased CaV3.1 T-type channel expression, which was restored by SAK3 treatment.

FIG. 10B shows the summary of CaV3.1 expression. Data are shown as mean±S.E.M., n=12 fields per animal, four animals per experimental condition. Scale bars: 200 μm.

FIGS. 100 and 10D show FOXP2 expression was markedly reduced in gRNA TAF1 edited animals and again normalized by SAK3 treatment. FIG. 10D is a summary of data shown in FIG. 10C.

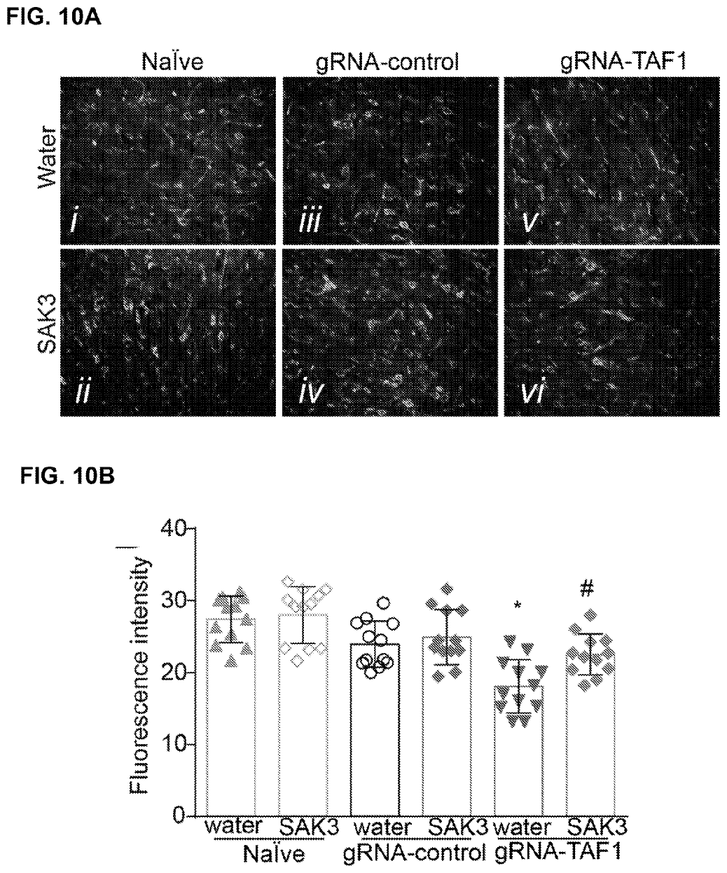

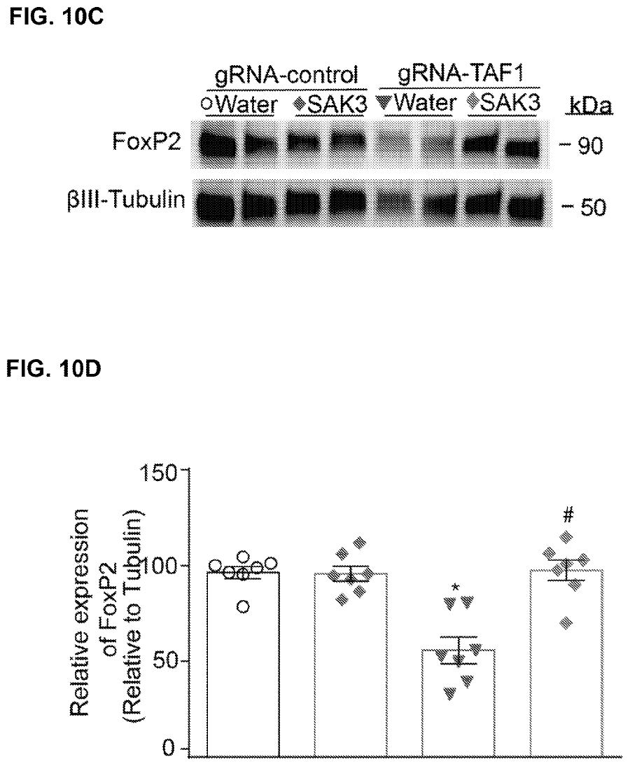

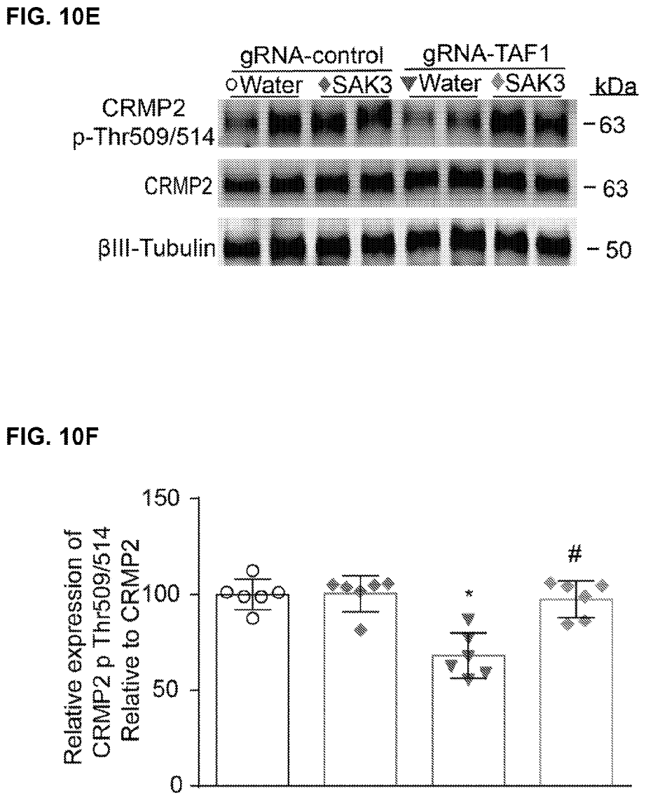

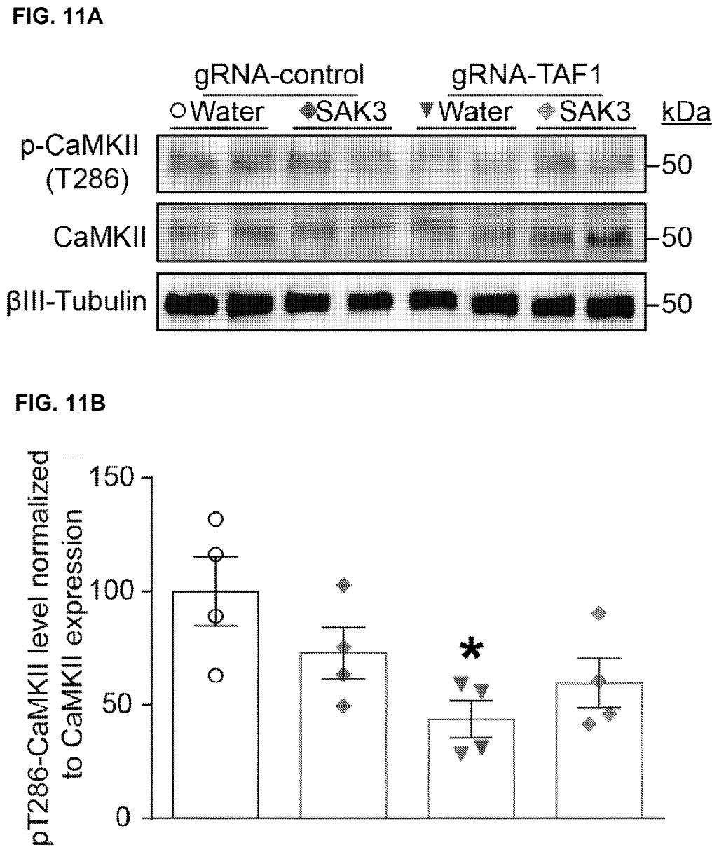

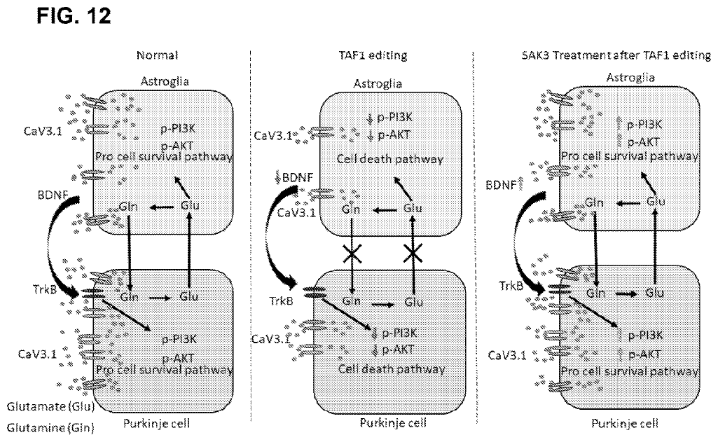

FIG. 10E shows p-CRMP2 levels were suppressed in gRNA TAF1 edited animals. However, SAK3 returned the phosphorylation levels of CRMP2, at Thr514 to control levels.

FIG. 10F shows the quantification of Western analysis from three independent experiments. Data shown are mean±SEM, n=6 animals per each experimental condition. *p<0.05 versus gRNA-control water and RNA-control SAK3 group; #p<0.05 versus gRNA-TAF1 water group (ANOVA followed by Tukey's test).

FIG. 11A shows a representative Western analysis is shown from cerebellum samples from each of the experimental conditions. Note decreased p-CaMKII in gRNA-TAF1 edited animals and increased p-CaMKII levels in gRNA-control.

FIG. 11B shows the quantification of p-CaMKII expression on all the experimental conditions. Data shown are mean+SEM, n=4 animals per each experimental condition. No significant changes were observed between the gRNA-TAF1 edited animals and SAK3 treated gRNA-TAF1 animals (Data Analyzed by Kruskal Wallis Test).

FIG. 12 shows a graphical representation of TAF1 editing affecting the calcium channel influx and cell survival pathway. SAK3 treatment enhances the Calcium channel receptor and cell survival pathway.

### DETAILED DESCRIPTION OF THE INVENTION

Before the present compounds, compositions, and/or methods are disclosed and described, it is to be understood that this invention is not limited to specific synthetic methods or to specific compositions, as such may, of course, vary. It is also to be understood that the terminology used herein is for the purpose of describing particular embodiments only and is not intended to be limiting.

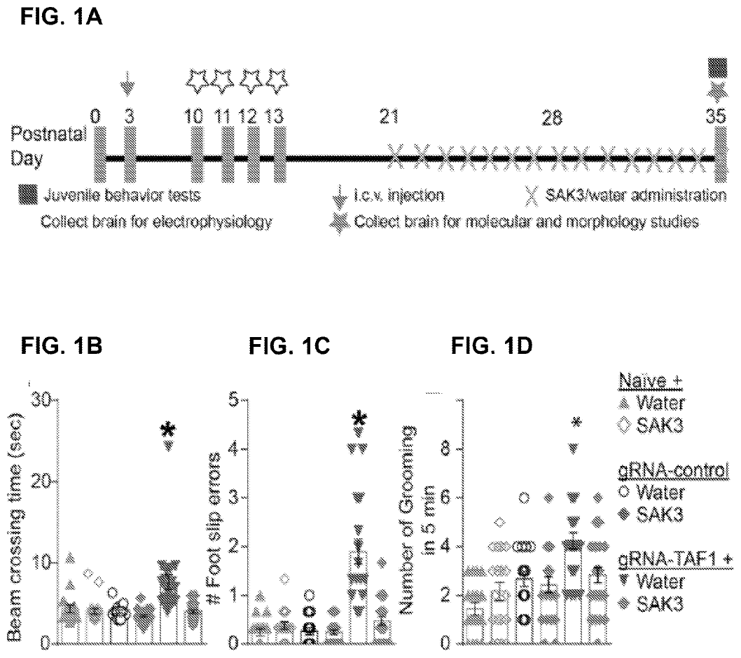

Referring now to FIGS. 1A-12, the present invention features a method of treating, preventing, and/or improving motor deficits associated with TAF1 ID Syndrome in a subject in need of such treatment.

According to one embodiment, the present invention may feature a method of treating motor deficits associated with TAF1 ID Syndrome in a subject in need of such treatment. In some embodiments, the method comprises administering a therapeutic amount of SAK3. The method may be capable of treating motor deficits associated with TAF1 ID Syndrome such that clinical improvements are observed.

In some embodiments, the present invention may feature a method of preventing motor deficits associated with TAF1 ID Syndrome in a subject in need of such treatment. In some embodiments, the method comprises administering a therapeutic amount of SAK3. In other embodiments, the present invention features a method of improving motor deficits and behavioral defects associated with TAF1 Intellectual Disability Syndrome (TAF1 ID) in a subject in need of such treatment. In some embodiments, the method comprises administering a therapeutic amount of SAK3 to the subject. In some embodiments, the subject is diagnosed with TAF1 ID Syndrome through genetic testing in early childhood.

In another embodiment, the present invention may feature a method of restoring protein levels and regenerating cells in vivo by administering SAK3. In some embodiments, the protein restored may be Forkhead box protein P2 (FOXP2) or Brain-derived neurotrophic factor (BDNF). In another embodiment, the cells regenerated are Purkinje cells or astrocytes from the cerebral cortex or the cerebellum.

As defined herein, the term “motor deficits” may refer to partial or total loss of function of a body part, usually a limb or limbs. This may result in muscle weakness, poor stamina, lack of muscle control, or total paralysis. In some embodiments, “motor deficits” may refer to beam crossing time or the number of foot slip errors.

In some embodiments, the methods described herein treat and/or improve neurological cognition. As used herein, “neurological cognition” here refers to mental activity and processes necessary for motor movement, thought, perception, and learning.

As defined herein, the term “regenerating” refers to replacing or restoring damaged or missing cells.

As defined herein, the term “TAF1 ID Syndrome” may also be known as mental retardation, X-linked, syndromic-33 disease, or X-linked syndromic mental retardation-33 (MRXS33, Online Mendelian Inheritance in Man (OMIM: 300966)). As defined herein the term “TAF1 ID Syndrome” occurs mainly in males, leading to abnormalities in global developmental (motor, cognitive, and speech), hypotonia, gait abnormalities, and cerebellar hypoplasia.

As used herein, SAK3 may refer to ethyl 8′-methyl-2′, 4-dioxo-2-(piperidin-1-yl)-2′H-spiro [cyclopent[2]ene-1,3′-imidazo[1,2-a]pyridine]-3-carboxylate. In some embodiments, SAK3 refers to a novel T-type Ca2+ channel enhancer.

As used herein, “clinical improvement” may refer to a noticeable reduction in the symptoms of a disorder or cessation thereof.

In some embodiments, clinical improvement may refer to an improvement of motor deficits such as balance. In other embodiments, clinical improvement may refer to an improvement of behavioral function, such as grooming. As used herein, “grooming” may refer to licking the fur, washing face, or scratching in rats. In further embodiments, clinical improvement may refer to the improvement of cognition and motor function. In other embodiments, clinical improvement may refer to the improvement of neurological cognition and motor deficits.

In one embodiment, the subject may be a mammal, such as a human. In another embodiment, the composition is administered in a dosage of about 0.1 mg to 1000 mg. For example, the dosage may range from 0.1 mg to 1000 mg with a preferred range of about 10 mg to 500 mg. The composition may be administered once daily or twice daily; or the composition may be administered at least once daily, at least once every other day, or at least once weekly or once monthly or administered for an extended period. In further embodiments, the composition may be administered orally, intravenously, or transdermally.

In some embodiments, the composition for use may be administered once daily or twice daily. In another embodiment, the composition may be administered at least once daily, at least once every other day, or at least once weekly or once monthly. Further still, the composition may be administered intravenously, transdermally, or orally. In preferred embodiments, the composition for use in the treatment resulted in clinical improvement of motor deficits associated with TAF1 ID Syndrome. For example, clinical improvement may be observed in about 1 to 7 days, or about 7 to 14 days, or about 1-3 months.

In any of the aforementioned embodiments of the present invention, the composition may be administered in a dosage of about 0.1 mg to 1000 mg. For example, the dosage may range from about 0.1 mg to 1 mg, 1 mg to 10 mg, 10 mg to 20 mg, 20 mg to 30 mg, 30 mg to 40 mg, 40 mg to 50 mg, 50 mg to 60 mg, 60 mg to 70 mg, 70 mg to 80 mg, 80 mg, to 90 mg, 90 to 100 mg, 100 mg to 200 mg, 200 mg to 300 mg, 300 mg to 400 mg, 400 mg to 500 mg. 500 mg to 600 mg, 600 mg to 700 mg, 700 mg to 800 mg, 800 mg to 900 mg, or 900 mg to 1000 mg.

Without wishing to limit the invention to a particular theory or mechanism, it is thought that SAK3 helps to mitigate motor deficits associated with TAF1 ID syndrome by restoring Ca²⁺ influx into cells, influencing glutamatergic pathways, and activating pro-survival cell pathways.

A “subject” is an individual and includes, but is not limited to, a mammal (e.g., a human, horse, pig, rabbit, dog, sheep, goat, non-human primate, cow, cat, guinea pig. or rodent), a fish, a bird, a reptile or an amphibian. The term does not denote a particular age or sex. Thus, adult, and newborn subjects, as well as fetuses, whether male or female, are intended to be included. A “patient” is a subject afflicted with a disease or disorder.

The present invention may feature a composition comprising a therapeutic amount of SAK3 for use in a method of treating TAF1 Intellectual Disability Syndrome (TAF1 ID). The present invention may also feature a composition comprising a therapeutic amount of SAK3 for use in a method of treating motor deficits associated with TAF1 Intellectual Disability Syndrome (TAF1 ID). The present invention may further feature a composition comprising a therapeutic amount of SAK3 for use in a method of preventing motor deficits associated with TAF1 Intellectual Disability Syndrome (TAF1 ID). The present invention may also feature a composition comprising a therapeutic amount of SAK3 for use in a method of restoring protein levels and regenerating cells in vivo.

In some embodiments, the cells are Purkinje cells. In some embodiments, the cells are astrocytes. In some embodiments, the protein is Forkhead box protein P2 (FOXP2). In other embodiments, the protein is Brain-derived neurotrophic factor (BDNF).

Disclosed are the various compounds, solvents, solutions, carriers, and/or components to be used to prepare the disclosed compositions as well as the compositions themselves to be used within the methods disclosed herein. Also disclosed are the various steps, elements, amounts, routes of administration, symptoms, and/or treatments that are used or observed when performing the disclosed methods, as well as the methods themselves. These and other materials, steps, and/or elements are disclosed herein, and it is understood that when combinations, subsets, interactions, groups, etc. of these materials are disclosed, that while specific reference of each various individual and collective combination and permutation of these compounds may not be explicitly disclosed, each is specifically contemplated and described herein.

The terms “administering” and “administration” refer to methods of providing a pharmaceutical preparation to a subject. Such methods are well known to those skilled in the art and include, but are not limited to, administering the compositions orally, parenterally (e.g., intravenously and subcutaneously), by intramuscular injection, by intraperitoneal injection, intrathecally, transdermally, extracorporeally, topically, or the like.

A composition can also be administered by topical intranasal administration (intranasally) or administration by inhalant. As used herein, “topical intranasal administration” means delivery of the compositions into the nose and nasal passages through one or both of the nares and can comprise delivery by a spraying mechanism (device) or droplet mechanism (device), or through aerosolization of the composition. Administration of the compositions by inhalant can be through the nose or mouth via delivery by a spraying or droplet mechanism. As used herein, “an inhaler” can be a spraying device or a droplet device for delivering a composition comprising SAK3, in a pharmaceutically acceptable carrier, to the nasal passages and the upper and/or lower respiratory tracts of a subject. Delivery can also be directed to any area of the respiratory system (e.g., lungs) via intratracheal intubation. The exact amount of the compositions required will vary from subject to subject, depending on the species, age, weight, and general condition of the subject, the severity of the disorder being treated, the particular composition used, its mode of administration, and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves the use of a slow-release or sustained-release system such that a constant dosage is maintained. See, for example, U.S. Pat. No. 3,610,795, which is incorporated by reference herein.

As used herein, the terms “treat”, “treating”, or “treatment” refer to both therapeutic treatment and prophylactic or preventative measures, with the objective of preventing, reducing, slowing down (lessen), inhibiting, or eliminating an undesired physiological change, symptom, disease, or disorder, such as TAF ID Syndrome. For purposes of this invention, beneficial or desired clinical results include, but are not limited to, alleviation of symptoms, diminishment of the extent of disease, stabilized (i.e., not worsening) state of disease, delay or slowing of disease progression, amelioration, or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented or onset delayed. Optionally, the subject or patient may be identified (e.g., diagnosed) as one suffering from the disease or condition (e.g., TAF ID Syndrome) prior to administration of the peptide analogue of the invention. Subjects at risk for TAF ID Syndrome can be identified by, for example, any or a combination of appropriate diagnostic or prognostic assays known in the art.

A “therapeutically effective amount” refers to an amount that is sufficient to achieve the desired therapeutic result or to have an effect on undesired symptoms but is generally insufficient to cause intolerable adverse side effects. The specific therapeutically effective dose level for any particular patient will depend upon a variety of factors, including the disorder being treated and the severity of the disorder; the specific composition employed; the age, body weight, general health, sex, and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of the composition at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary and can be administered in one or more dose administrations daily, for one or several days. Guidance can be found in the literature for appropriate dosages for given classes of pharmaceutical products.

As described above, the compositions can be administered to a subject in a pharmaceutically acceptable carrier. By “pharmaceutically acceptable” is meant a material that is not biologically or otherwise undesirable, i.e., the material may be administered to a subject without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

Pharmaceutical carriers are known to those skilled in the art. These most typically would be standard carriers for the administration of drugs to humans, including solutions such as sterile water, saline, and buffered solutions at physiological pH. Typically, an appropriate amount of a pharmaceutically acceptable salt is used in the formulation to render the formulation isotonic. Examples of the pharmaceutically acceptable carrier include, but are not limited to, saline, Ringer's solution, and dextrose solution. The pH of the solution is preferably from about 5 to about 8, and more preferably from about 7 to about 7.5. Further carriers include sustained release preparations such as semi-permeable matrices of solid hydrophobic polymers containing the disclosed compounds, which matrices are in the form of shaped articles, e.g., films, liposomes, microparticles, or microcapsules. It will be apparent to those persons skilled in the art that certain carriers can be preferable depending upon, for instance, the route of administration and concentration of composition being administered. Other compounds can be administered according to standard procedures used by those skilled in the art.

Pharmaceutical formulations can include additional carriers, as well as thickeners, diluents, buffers, preservatives, surface-active agents, and the like in addition to the compounds disclosed herein. Pharmaceutical formulations can also include one or more additional active ingredients such as antimicrobial agents, anti-inflammatory agents, anesthetics, and the like.

The pharmaceutical formulation can be administered in several ways depending on whether local or systemic treatment is desired, and on the area to be treated. A preferred mode of administration of the composition is orally. Other modes of administration may be topically (including ophthalmically, vaginally, rectally, intranasally), by inhalation, or parenterally, for example by intravenous drip, subcutaneous, intraperitoneal, or intramuscular injection. The disclosed compounds can be administered orally, intravenously, intraperitoneally, intramuscularly, subcutaneously, intracavity, transdermally, sublingually or through buccal delivery.

Pharmaceutical compositions for oral administration include, but are not limited to, powders or granules, suspensions or solutions in water or non-aqueous media, capsules, sachets, or tablets. Thickeners, flavorings, diluents, emulsifiers, dispersing aids, or binders may be desirable. The composition of SAK3 can be administered to a subject orally in a dosage taken once daily or in divided doses. A person of skill, monitoring a subject's clinical response, can adjust the frequency of administration of the medication according to methods known in the art.

Preparations for parenteral administration include sterile aqueous or non-aqueous solutions, suspensions, and emulsions. Examples of non-aqueous solvents are propylene glycol, polyethylene glycol, vegetable oils such as olive oil, fish oils, and injectable organic esters such as ethyl oleate. Aqueous carriers include water, alcoholic/aqueous solutions, emulsions, or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, antioxidants, chelating agents, and inert gases, and the like.

Pharmaceutical formulations for topical administration may include ointments, lotions, creams, gels, drops, suppositories, sprays, liquids, and powders. Conventional pharmaceutical carriers, aqueous, powder or oily bases, thickeners, and the like may be necessary or desirable.

In one aspect, SAK3 can be administered in an intravenous dosage. This dosage can be administered to a subject once daily or in divided doses throughout a day, as determined by methods known in the art. This dosage can be administered to a subject daily until a clinical response is noted. It is contemplated that the dosage of SAK3 can be administered as infrequently as once daily or weekly, or at any interval in between, depending on a subject's clinical response to the medication. If a subject does not respond to the initial dosage and administration of SAK3, a person of skill can administer the medication daily for several days until such response occurs. A person of skill can monitor a subject's clinical response to the administration of SAK3 and administer additional dosages if the subject's sleep disruption symptoms reappear after a period of remission. It is contemplated that SAK3 can be administered to a subject with, for example, motor deficits associated with TAF1 ID syndrome on a twice daily basis, once daily basis, on an alternating daily basis, on a weekly basis, on a monthly basis, or at any interval in between.

In another aspect, SAK3 can be administered to a subject transdermally, by using an adherent patch, by using iontophoresis, or by using any other method known to a person of skill. The dosage of SAK3 administered transdermally can be given daily or weekly, or at any interval in between. A person of skill, monitoring a subject's clinical response and improvement, can determine the frequency of administration of the medication by methods known in the art.

In another aspect, SAK3 can be administered to a subject intranasally in a dosage taken once daily or in divided doses. The medication can be administered daily or weekly, or at any interval in between. A person of skill, monitoring a subject's clinical response to the administration of the medication, can adjust the frequency of administration according to methods known in the art.

In another aspect, SAK3 can be administered to a subject intramuscularly in a dosage taken once daily or in divided doses. The medication can be administered daily or weekly, or at any interval in between. A person of skill, monitoring a subject's clinical response, can adjust the frequency of administration of the medication according to methods known in the art.

In some embodiments, the present invention may be used in combination with other treatments.

### EXAMPLES

The following are non-limiting examples of the present invention. It is to be understood that said example is not intended to limit the present invention in any way. Equivalents or substitutes are within the scope of the present invention.

Example 1 describes how SAK3 helps to improve motor deficits in a TAF1 ID Syndrome model.

Pathogen-free, normal E18 pregnant Sprague—Dawley rats (Envigo Laboratories) were housed 1 per cage in temperature- (23±3° C.) and light (12-h light/12-h dark cycle; lights on 07:00-19:00)-controlled rooms with standard rodent chow and water available ad libitum. The neonates are designated as postnatal day 0 (PDO) on the day of birth; the litter size range included in the current study was 11-14 pups.

After weaning, the rat pups were separated from the dams and maintained 4 per cage. Animals were divided into six groups. SAK3 (Catalog No: SML-2039-5MG, Sigma Aldrich) was dissolved in distilled water and orally administered (0.25 mg/kg, p.o) to the animals from PD21 to PD35 (FIG. 1A). Rats were sacrificed at PD35. Animals were behaviorally assessed before being euthanized for histological and protein expression analysis. All biochemical, electrophysiology, and behavior experiments were performed in a blinded fashion. Animal protocols were approved by the Institutional Animal Care and Use Committee of the College of Medicine at the University of Arizona and conducted in accordance with the Guide for Care and Use of Laboratory Animals published by the National Institutes of Health.

Intracerebroventricular injections: Bilateral intracerebroventricular (ICV) injections were performed as previously described in Sprague-Dawley (SD) rat pups on postnatal day 3. Briefly, newborn SD rat puts were anesthetized by isoflurane. A 10 μl syringe (Hamilton Gastight Syringe, #1701) was used to pierce the skull (coordinates from bregma: −0.6 mm posterior, ±1.75 mm lateral/medial, and −2.5 mm ventral), and 2.5 μl of CRISPR lentivirus (gRNA-control or gRNA-TAF1) was injected into each cerebral ventricle without opening the scalp. Neonatal rat pups were kept with the dam until weaned.

Cerebellar Slice Preparation: Rat pups were sacrificed between postnatal day 10 to 14 for electrophysiological analyses. The animals were decapitated after being deeply anesthetized with isoflurane, and their cerebellums were rapidly removed and placed in an ice-cold dissection ACSF containing (in mM) 220 sucrose, 2.5 KCl, 1.25 Na2HPO4, 3.5 MgCl2, 0.5 CaCl2, 25 NaHCO3, and 20 D-glucose (with pH at 7.4 and osmolarity at 310 mOsm), bubbled with 95% O2 and 5% CO2. Parasagittal slices (320 μm thick) were cut using a VT 1200S vibratome (Leica, Germany). Slices were then incubated for at least 1 hour at 34° C. in an oxygenated recording solution containing (in millimolar): 125 NaCl, 2.5 KCl, 2 CaCl2, 1 MgCl2, 1.25 NaH2PO4, 26 NaHCO3, 25 D-glucose, with pH at 7.4 and osmolarity at 320 mOsm. The slices were then positioned in a recording chamber and continuously perfused with oxygenated recording solution at a rate of 2 to 3 mL/min before electrophysiological recordings at RT.

Immunohistochemistry: The antibodies used in this study are listed in Table 1. Rats (n=6 animals per experimental condition) were perfused with saline, and 4% formaldehyde in phosphate buffer saline (PBS) at PD 35, and the brains were extracted and post-fixed in 4% paraformaldehyde for 8 hours at 4° C. Cerebral cortex sections were cut sagittally at 20 μm using a cryostat (Microm HM 505 E). After rinsing the sections in PBS for 5 min, the sections were incubated with a 0.1% H2O2 solution in PBS for 5 min, rinsed in PBS for 5 times for 5 min, and incubated for 30 min with 0.4% Triton X-100, rinsed in PBS and blocked with 8% goat serum and 1% Triton X-100 in PBS. After blocking, the sections were incubated at 4° C. overnight with the indicated antibodies diluted in 4% goat serum in PBS. The sections were washed in 1% goat serum in PBS, incubated with secondary antibody anti-rabbit Alexa fluor 488 (Life Technologies) or anti-mouse Alexa fluor 488 (Life Technologies), as needed, in 4% goat serum for 2 h, washed with PBS 3 times for 5 mins, and incubated with DAPI (Catalogue No: D1306, Thermofisher Scientific) at a concentration of 50 ng/ml for 2 mins. Sections were then washed and further air dried, and coverslip with glycerol. All procedures were performed at room temperature. stained slides were observed under a fluorescence microscope (LSM510, Carl Zeiss) using a 20× objective and apotome 2. Immunofluorescence was quantified in 12 different fields from 4 different animals per experimental as previously described in Janakiraman, U., et al. (TAF1-gene editing alters the morphology and function of the cerebellum and cerebral cortex. Neurobiol Dis, 2019. 132: p. 104539).

Cell Counting: Twelve visual fields (0.6 mm²) of the cerebral cortex were randomly imaged from each section. The number of stained cells in each field was counted at a 40× magnification. Data were expressed as the number of cells per field.

Morphometric analysis: We quantified the number of calbindin+, GFAP+, Iba1a+, and TUNEL+ cells with the granular layer from 12 fields from 4 different animals per experimental condition. The immunofluorescence for BDNF and pS473-AKT was quantitated in 12 different fields of cerebellar tissue from 4 different animals per experiment in a similar fashion.

Statistics: All experiments were performed at least twice and in a blinded fashion. All data were first tested for a Gaussian distribution using a D'Agostino-Pearson test (Graphpad Prism 8 Software). The statistical significance of differences between means was determined by a parametric ANOVA followed by Tukey's post hoc or a non-parametric Kruskal Wallis test followed by Dunn's post-hoc test depending on whether datasets achieved normality. Differences were considered significant if p≤0.05. Error bars in the graphs represent mean±SEM. All data were plotted in GraphPad Prism 8.

SAK3 Improves TAF1 ID-Like Motor Deficits

Open Field Test: The apparatus (W100 cm×D100 cm×H40 cm) is made of wood. The floor of this chamber was divided into 25 cm (5×5) squares. The rat pups were placed into one corner of an open field chamber and their behavior was observed for 5 min. The number of groomings, i.e. consisting of licking the fur, washing face, or scratching behaviors were noted.

Beam Walking Test: Animals were allowed to walk on a narrow flat stationary wooden beam (L100 cm×W2 cm) placed at a height of 100 cm from the floor to reach an enclosed escape platform. The time taken to cross the beam from one end to the other and the number of foot slip errors were observed as described previously in Manivasagam et al, 2009.

Motor dysfunction is characteristic of TAF 1D syndrome. TAF1-edited animals have concomitant CaV3.1 loss with motor deficits and abnormal grooming behaviors. Therefore, the present invention sought to determine whether the T-type Ca2+ channel enhancer, SAK3, could mitigate these behavioral abnormalities in TAF1-edited animals (FIG. 1A). Beam crossing time (FIG. 1B) and the number of foot slip errors were higher in the TAF1-edited animals compared to all other experimental groups (FIG. 1C). However, in TAF1-edited animals treated with SAK3, there was a rescue of the beam crossing time and foot slip errors compared to TAF1-edited animals. TAF1-edited animals displayed an increased grooming frequency which was blunted by SAK3 treatment (FIG. 1D). TAF1-edited animals treated with SAK3 showed similar behavioral patterns as the naïve group. Thus, enhancing T-type Ca2+ channels using SAK3, rescued the behavioral defects associated with TAF1 editing.

SAK3 Attenuates the Morphological Abnormalities Caused by TAF1 Deletion

Nissl Staining: After dehydration of the tissue with 30% sucrose, 20 μm sections were cut, stained with cresyl violet dye (Catalog No: 05042-10G, Sigma), and mounted with Richard-Allan scientific mounting medium (Catalog No: 4112, Thermo Scientific) for microscopy.

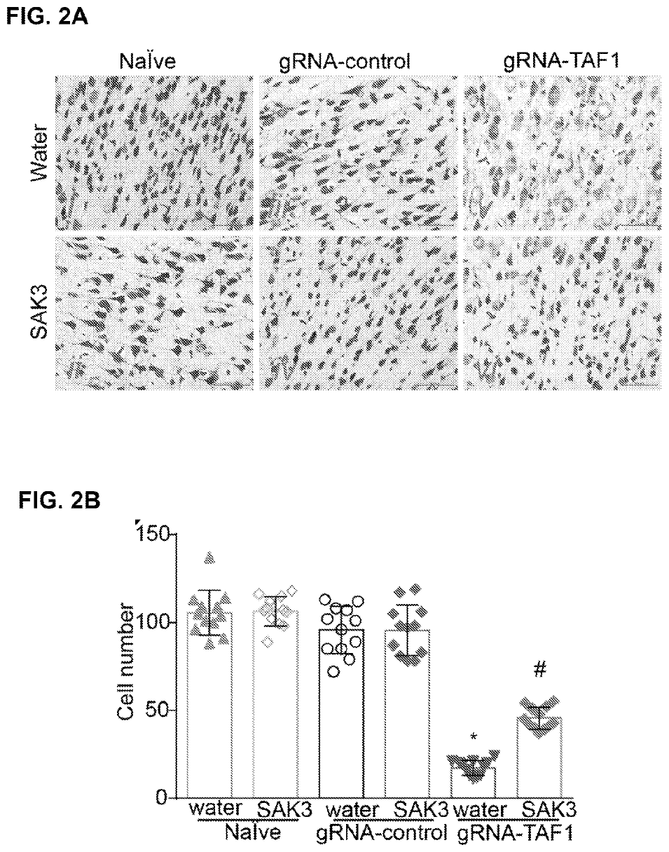

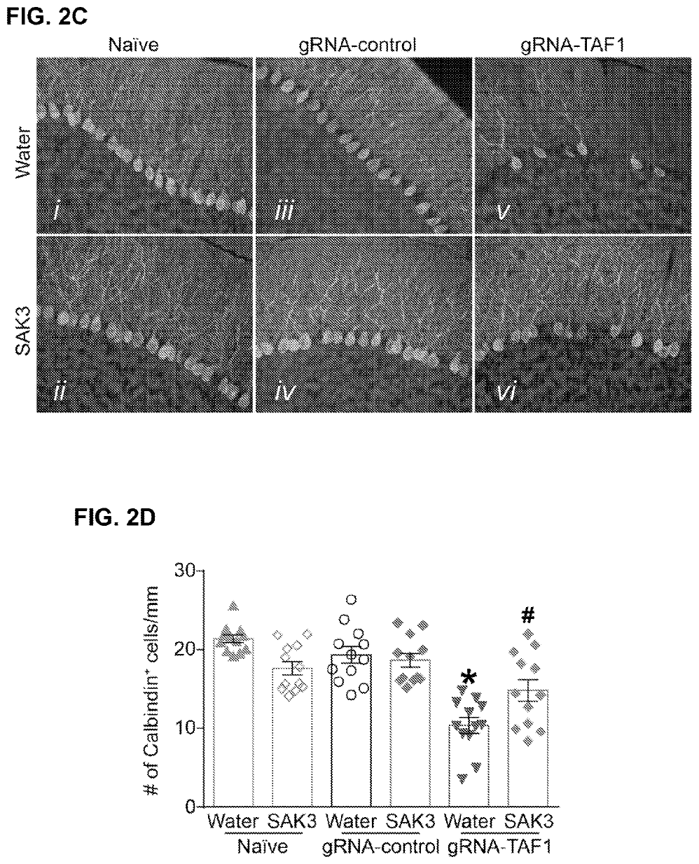

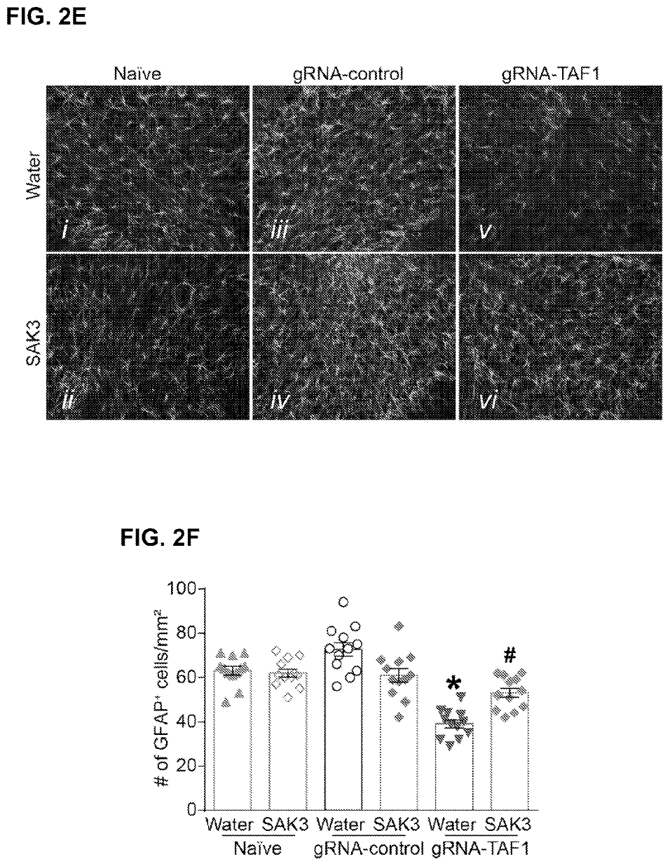

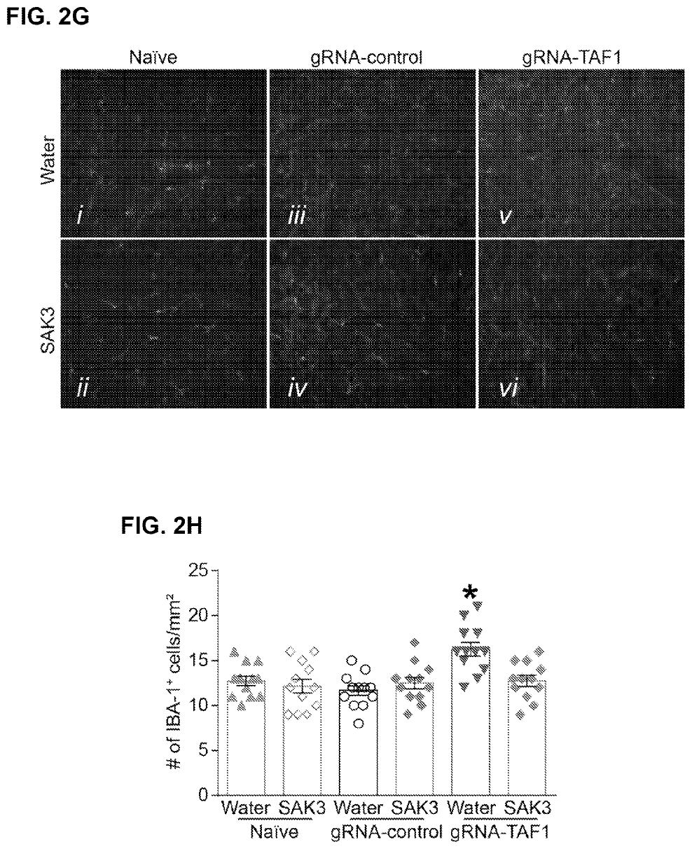

TAF1-editing causes morphological abnormalities in the cells of the cerebral cortex, so the morphology of the cerebral cortex neurons was evaluated by Nissl staining. The histopathology analysis showed that neurons in the control group were arranged tightly with regular morphology and had an intact cell structure. The blue Nissl bodies in the neurons were visible and distinct. The morphology of the cells in the cortex clearly changed upon TAF1-editing. The shape of the cells changed from pyramidal to stellate morphology and a large number of cells appeared to be vacuolated with enlarged intercellular spaces. Also, the cells did not stain as distinctly as control cells with the Nissl dye (FIG. 2A). However, SAK3 administration improved the morphology of the cells in the cortex (FIG. 2A). Next, the viability of these cells was determined. TAF-1 editing reduced the number of viable cells compared to Naïve or gRNA-control rats (FIG. 2B). SAK3 administration increased the number of viable cells compared to TAF1-edited animals (FIG. 2B). Treatment with SAK3 had no effect on the Naïve and gRNA-control group compared to the vehicle (water) (FIG. 2B). Collectively, these results strongly suggest that TAF1 is directly implicated in neuronal survival.

Next, whether SAK3 could prevent the loss of Purkinje cells observed after TAF1 editing was assessed (FIG. 2C and FIG. 2D). Rat cerebella were stained with the Purkinje cells marker Calbindin and quantified the number of Purkinje cells per linear mm. As before, TAF1 editing reduced the number of Purkinje cells compared to Naïve or gRNA-control rats. SAK3 administration increased the number of calbindin-positive Purkinje cells compared to TAF1-edited animals (FIG. 2D). Treatment with SAK3 had no effect on the Naïve and gRNA-control group compared to the vehicle (water) (FIG. 2B).

Glial cells, including astrocytes, oligodendrocytes, and microglia are by far the most abundant cells in the nervous system including in the granular layer of the cerebellum. In TAF1-edited animals, there is a decrease in GFAP-positive astrocytes and an increase in Iba1-positive microglia within the granular layer of the cerebellum. In the present study, these findings were confirmed (FIG. 2E). SAK3 treatment was found to increase the number of GFAP-positive cells (FIG. 2F) and decrease the number of Iba1-positive cells compared to the TAF1 edited group (FIG. 2G and FIG. 2H). Thus, enhancing T-type Ca2+ channel activity with SAK3 can reverse all morphological alterations induced by TAF1 editing.

SAK3 Rescued the Decreased Frequency Of Spontaneous Excitatory Postsynaptic Current (sEPSCs) in TAF1 Edited Purkinje Cells

Whole-Cell Patch-clamp Recording: Recordings were made from PCs in lobules IV-VI, which were visually identified based on their location using infrared differential interference contrast video microscopy on an upright microscope (FN1; Nikon, Tokyo, Japan) equipped with a 3 40/0.80 water-immersion objective and a charge-coupled device camera. The pipettes were prepared by pulling glass capillaries on a model p-97 microelectrode puller (Sutter Instrument, Novato, USA). Patch pipettes had resistances of 3-5 MΩ. The internal solution was a K-based solution containing (in mM): 120 potassium gluconate, 20 KCl, 2 MgCl 2, 2 Na 2-ATP, 0.5 Na-GTP, 20 HEPES, 0.5 EGTA, with pH at 7.28 (with potassium hydroxide [KOH]) and osmolarity at 310 mOsm.

The whole-cell configuration was obtained in voltage-clamp mode. The membrane potential was held at −70 mV using PATCHMASTER software in combination with a patch clamp amplifier (EPC10; HEKA Elektronik, Lambrecht, Germany). To record spontaneous excitatory postsynaptic currents (sEPSCs), bicuculline methiodide (10 μM) was added to the recording solution to block γ-aminobutyric acid-activated currents. SAK3 (0.1 nM) was also added to the perfusion system. Hyperpolarizing step pulses (5 mV in intensity, 50 milliseconds in duration) were periodically delivered to monitor the access resistance (15-25 MΩ), and recordings were discontinued if the access resistance changed by more than 20%. For each PC, sEPSCs were recorded for a total duration of 2 minutes. Currents were filtered at 3 kHz and digitized at 5 kHz. Data were further analyzed by the Mini-Analysis (Synatosoft Inc, N.J.) and Clampfit 10.7 Program. The amplitude and frequency of sEPSCs were compared between neurons from three groups.

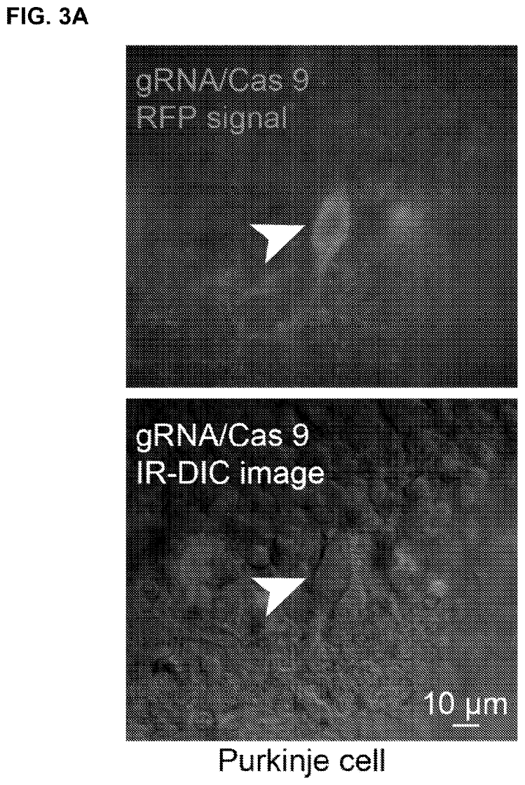

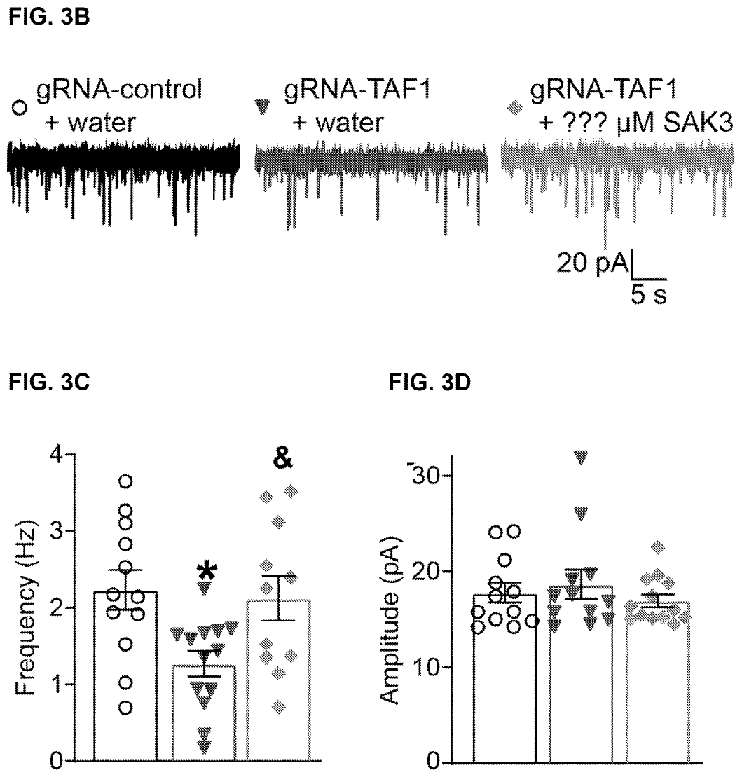

Abnormal motor symptoms in TAF1-edited rats are associated with irregular cerebellar output caused by changes in the intrinsic activity of the Purkinje cells due to loss of presynaptic Cav3.1. Therefore, if SAK3 could directly mitigate the defects in presynaptic currents in TAF1-edited animals was investigated. Spontaneous excitatory postsynaptic currents (sEPSCs) were recorded from gRNA-control and gRNA-TAF1 edited Purkinje cells within cerebellar slices (FIG. 3A) using whole-cell patch clamp electrophysiology (FIG. 3B). TAF1 editing decreased the frequency of sEPSC compared to gRNA-control (1.27±0.17 Hz vs 2.23±0.26 Hz, *p<0.05). Applying SAK3 (0.1 nM) rescued sEPSC frequency to the level of gRNA control (2.13±0.29 Hz, &p<0.05) (FIG. 3C). No changes in the amplitudes of sEPSC for any treatment groups were observed (FIG. 3D). These results show that SAK3 treatment can restore functional neurotransmission in gRNA-TAF1 edited Purkinje cells.

SAK3 Attenuates TAF1 Editing Induced Apoptosis

Western blotting analysis: After decapitation of animals, brain tissue from the cerebellum was dissected, snap frozen in liquid nitrogen prior to storage at −80° C. until analysis. Western blot analyses were performed as described in Janakiraman et al (Janakiraman, U., Yu, J., Moutal, A., Chinnasamy, D., Boinon, L., Batchelor, S. N., Anandhan, A., Khanna, R., and Nelson, M. A. (2019) TAF1-gene editing alters the morphology and function of the cerebellum and cerebral cortex. Neurobiol Dis 132, 104539). In brief, frozen samples were homogenized with a RIPA buffer and centrifuged at 4 oC, 20,000xg for 20 min. Supernatant protein concentrations were determined using BCA method (Catalog No: #23227, Thermo Scientific), and samples were then boiled 5 min in Laemmli's sample buffer (Catalog No: NP0008, Life Technologies). Equal amounts of protein were loaded onto and run on SDS-polyacrylamide gels (Catalog No: #4568084, Bio-Rad) and then transferred to polyvinylidene difluoride membrane (Catalog No: #1620177, Bio-Rad). After transfer, membranes were blocked with TBST solution (50 mM Tris-HCl, pH 7.5, 150 mM NaCl, and 0.1% Tween 20) containing 5% non-fat dry milk at 4 oC for 1 hour. After blocking membranes were incubated overnight at 4° C. with anti-CaMKII (pan) (1:500; Cell Signaling), anti-phospho-CaMKII (Thr-286) (1:1000; Cell Signaling), anti-cleaved caspase-3 (Asp175) (1:1000; Cell Signaling), anti-Bax (1:1000; Cell Signaling) anti-Bcl-2 (1:1000; Abcam), anti-phospho-GSK3p(Ser9) (1:1000; Cell Signaling), anti-GSK3p (1:1000; Cell Signaling), anti-BDNF (1:750; Abcam), anti-Foxp2 (1:1000; Abcam), anti-CRMP2 pThr509/Thr514 (1:1000; Kina Source Limited), anti-CRMP2 (1:1000; Sigma), and 6111-Tubulin (1:1000; Promega) in 3% BSA. After washing, membranes were incubated with secondary antibody diluted in 3% BSA. Blots were developed using an ECL detection system (Catalog No: 20-300B, Prometheus Protein Biology Products, USA) and signals were quantified using Image Studio Digits software version 5.2 (Li-Cor).

TUNEL Assay: Cerebral cortex tissue sections of 20 μm were cut and 5-7 sections chosen according to systematic random sampling scheme from each sample were processed with In Situ Cell Death Detection Kit, Fluorescin (Catalog No: 11684795910; Roche, Millipore Sigma, USA) according to the manufacturer's protocol for tissues. Then the slides were observed under a fluorescence microscope (LSM510, Carl Zeiss) using a 20× objective and apotome 2.

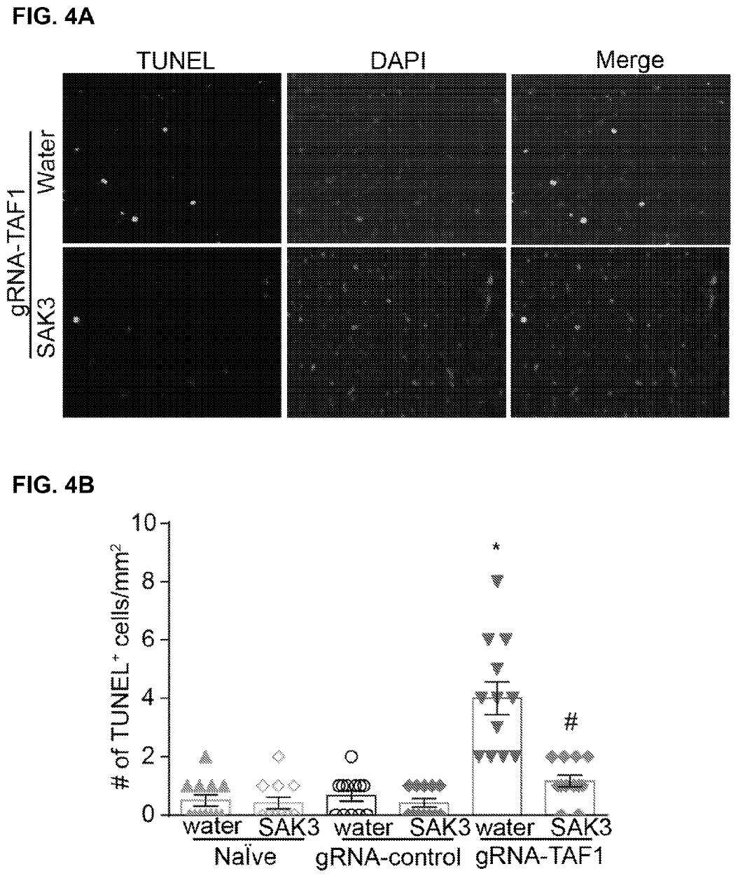

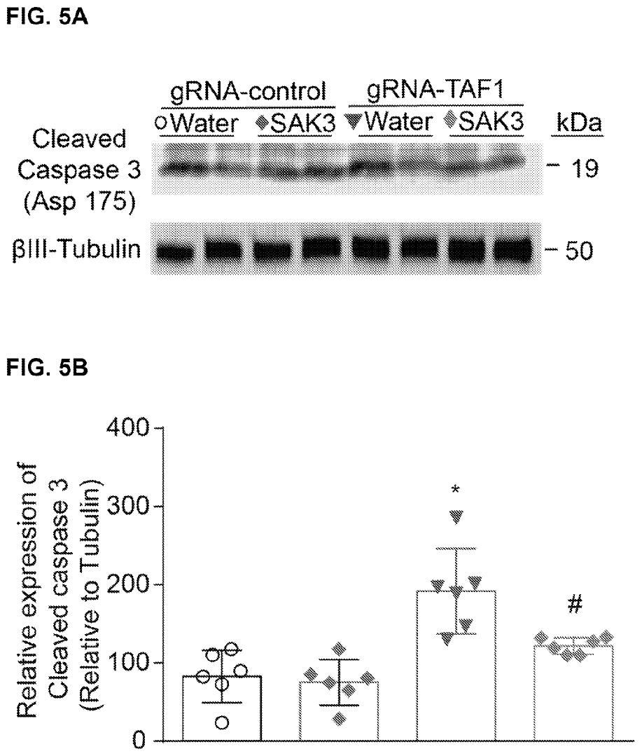

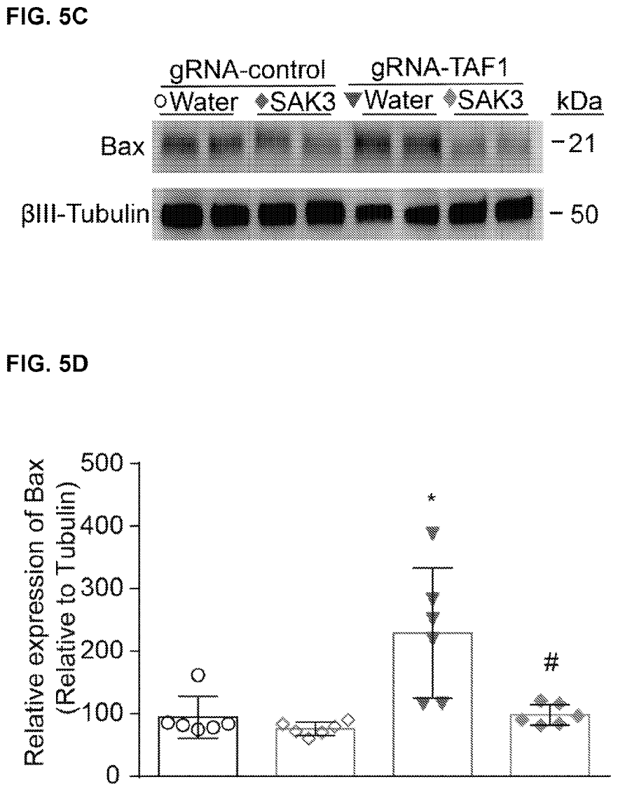

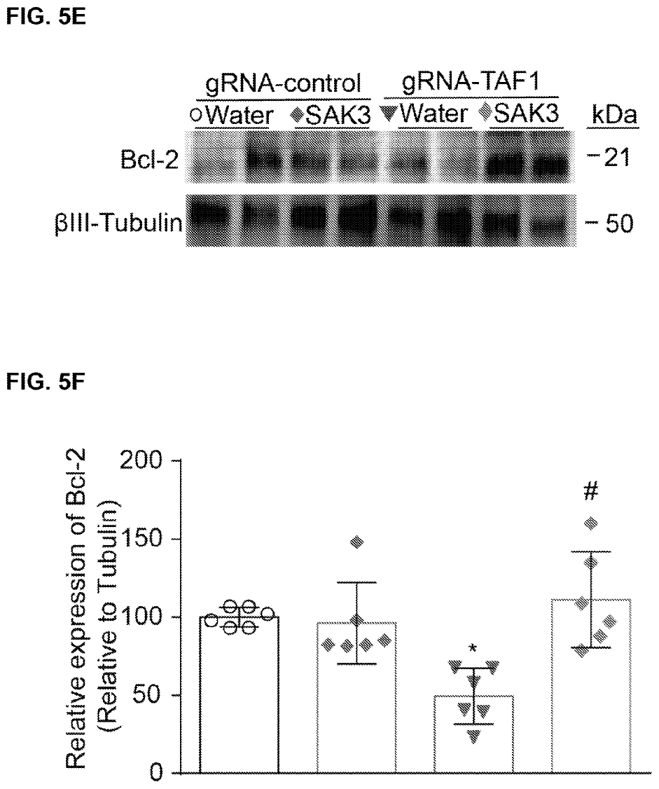

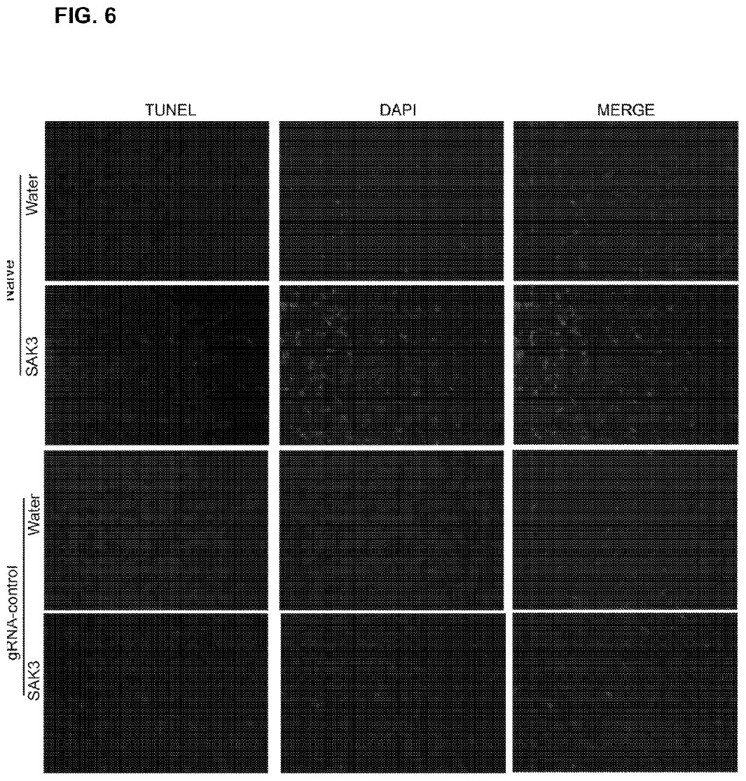

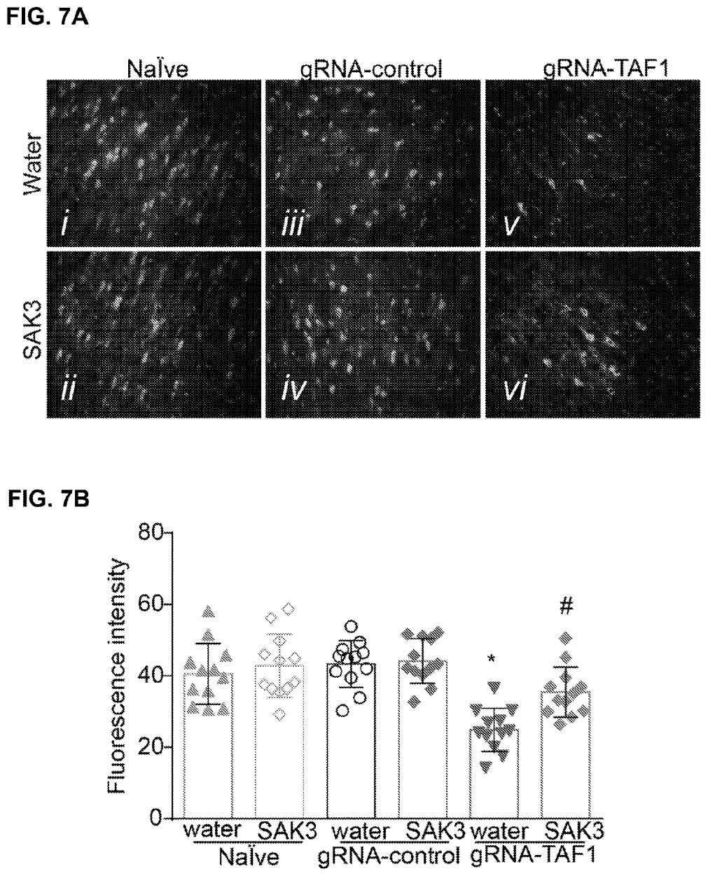

Because morphologic abnormalities and decreased cell viability were observed in TAF1 edited animals that were rescued by SAK3, apoptosis was assessed to determine if it could be enhanced in TAF1 edited animals. An increase in terminal deoxynucleotidyl transferase dUTP nick end labeling (TUNEL) positive cells in TAF1 edited animals. SAK3 treatment reduced the number of TUNEL positive cells to control levels in TAF1 edited animals (FIGS. 4A-4B). No significant changes in the TUNEL positive cells of water and SAK3 treatment to the naïve and gRNA-control animals were observed (FIG. 6). The protective effects of SAK3 against apoptosis were corroborated by (i) the downregulation of activated caspase 3, a protein responsible for the cleavage of many key proteins (FIGS. 5A-5B); (ii) the downregulation of the pro-apoptotic factor BCL2 Associated X (BAX), an apoptosis regulator (FIGS. 5C-5D); and (iii) up-regulation of B-cell lymphoma 2 (BcI-2), an apoptosis suppressor gene (FIGS. 5E-5F).

SAK3 Signaling in the Cerebral Cortex of TAF1 Edited Animals

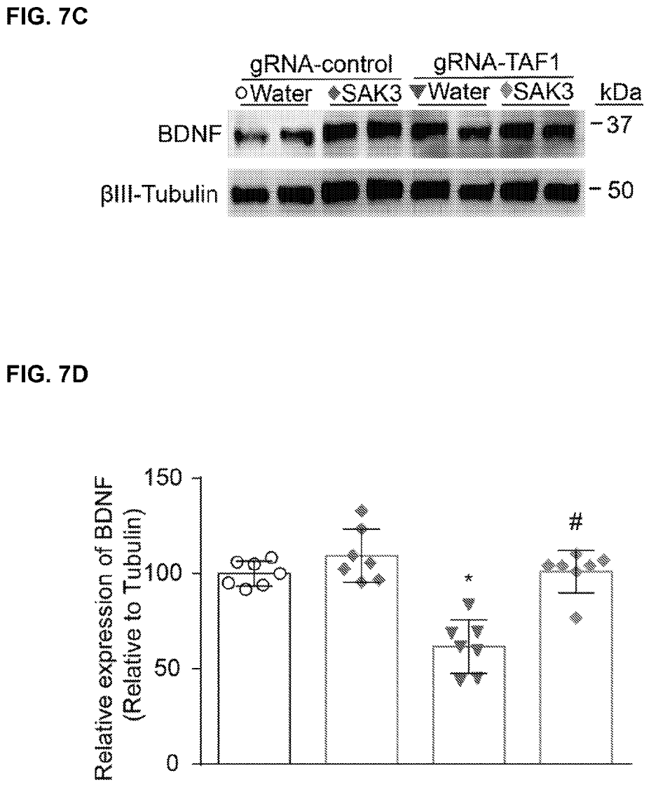

Brain-derived neurotrophic factor (BDNF) plays a crucial role in promoting the survival and differentiation of neurons. Activation of the PI3K/AKT pathway through BDNF/TrkB interactions can inhibit apoptosis in the developing cerebellum. Consequently, it was tested if SAK3 could be neuroprotective through this pathway. BDNF levels were markedly lower in TAF1 edited animals compared to Naïve and gRNA-control groups (FIGS. 7A-7D). However, SAK3 administration restored BDNF expression to the levels in Naïve and gRNA-control groups (FIGS. 7A-7D).

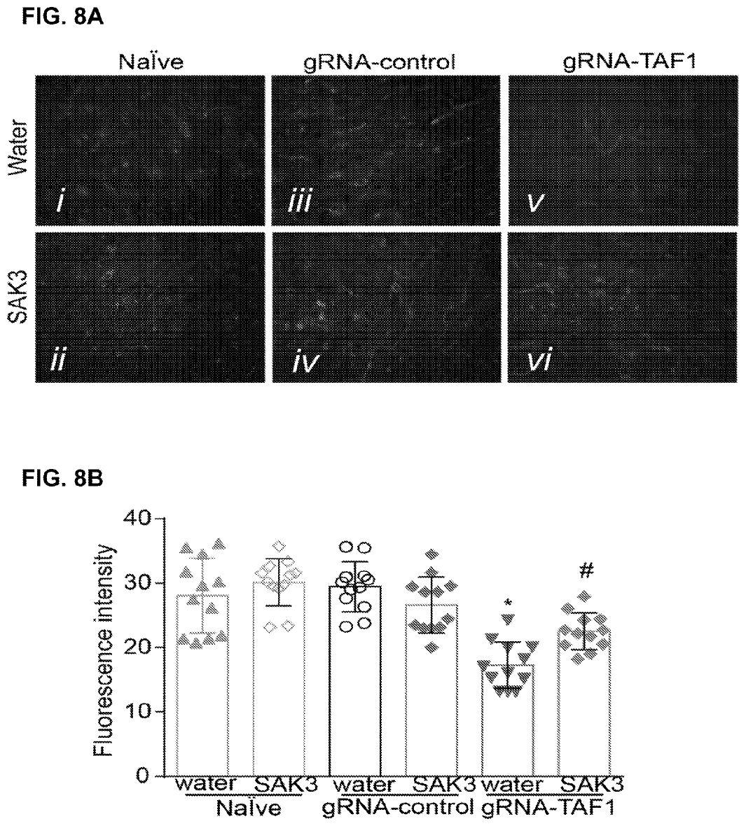

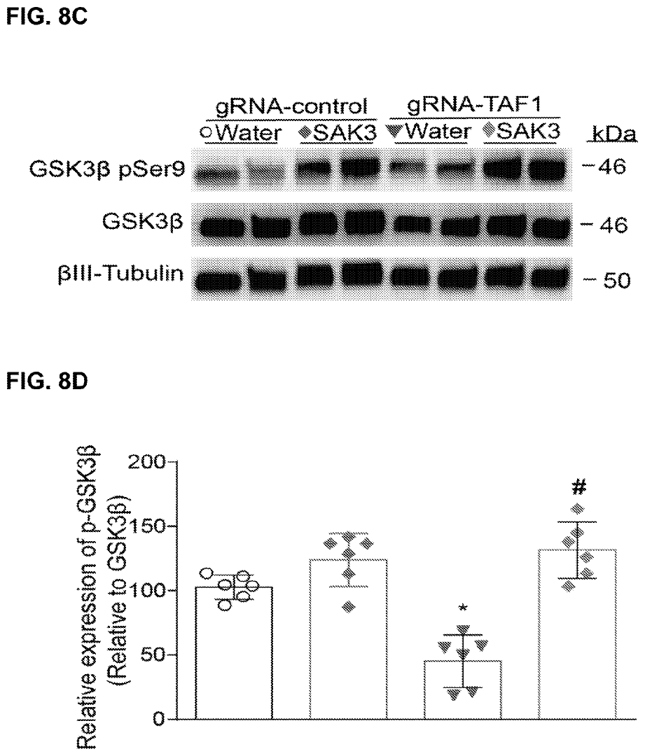

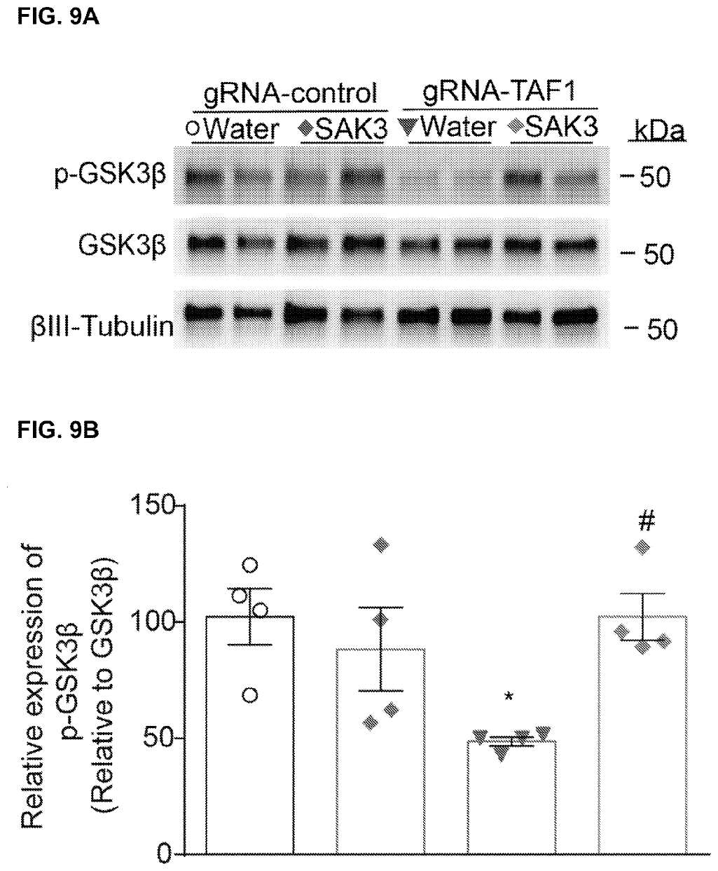

Protein kinase B (AKT) phosphorylation at Ser 473 is associated with its activation. Activated AKT (phosphorylated at Ser 473) was decreased in the cortex of TAF-1 edited animals which were prevented by SAK3 treatment (FIGS. 8A-8B). Glycogen synthase kinase 3 (GSK3) has two isoforms, GSK3a and GSK3β, that are inactivated by AKT phosphorylation on Ser 21 and Ser 9, respectively. Inhibition of GSK3β is known to protect against apoptosis in many situations. GSK3β is a downstream target of AKT and activation of AKT inhibits GSK3β by inducing its phosphorylation. Phospho (Ser 9)-GSK3β levels were decreased in TAF1 edited animals and SAK3 treatment increased phopho-GSK3β levels (FIGS. 8C-8D). Similar effects were seen in the cerebellum (FIGS. 9A-9B)

GSK3 is a highly evolutionarily conserved multifaceted ubiquitous enzyme with over 40 different downstream substrates. Three proteins known to be regulated by GSK3β were evaluated—(i) (CaV3.1, the transcription factor Forkhead box protein P2 (FOXP2), and (iii) the axonal guidance and growth cone collapsin response mediator protein 2 (CRMP2). All three are expressed and have functions in cortical neurons. TAF1 editing resulted in a reduction of CaMKII activation (measured by phosphorylation at T286) in the cerebellum (FIGS. 10A-10B & FIGS. 11A-11B). However, a restoration of CaMKII activation in the cerebellum of SAK3 treated animals was not observed (FIGS. 10A-10B & FIGS. 11A-11B). In regard to FOXP2, a suppression in FOXP2 levels in TAF1-edited animals was observed and SAK3 treatment restored FOXP2 (FIGS. 10C-10D). With respect to CRMP2, while there was no difference in total CRMP2 protein levels, phospho-CRMP2 (Thr 509/514) levels were decreased in TAF1 edited animals, whereas SAK3 once again restored phospho-CRMP2 levels (FIGS. 10E-10F).

It has been presently demonstrated that T-type channel enhancer SAK3 can efficiently reverse behavioral, morphological, and biochemical defects induced by TAF1 editing in neonatal rats. The following salient observations were made: (a) SAK3 improves behavioral defects associated with TAF1 gene editing; (b) SAK3 restores the number of Purkinje, astrocytes, and microglial cells in the developing cerebellum that were lost following TAF1 editing; (c) SAK3 protects the neurons of the cerebellum from the deleterious effects of TAF1 editing; (d) SAK3 restores excitatory postsynaptic current (sEPSCs) in TAF1 edited Purkinje cells; and (e) SAK3 activates BDNF/AKT/GSK36 signaling in TAF1 edited animals.

In conclusion, the present findings suggest that the T-type calcium channels are novel molecular targets to develop therapeutics to treat TAF1 ID syndrome and that SAK3 is an attractive drug candidate to treat TAF1 associated neurologic disorders.

Example 2 describes the treatment of motor deficits associated with TAF1 ID Syndrome involving oral administration of SAK3.

A one-year-old child is brought by his mother to see his pediatrician for a routine childhood check-up. During the check-up, the pediatrician notices some minor motor deficits in the child. The pediatrician communicates her concerns about the child to his mother. The pediatrician suggests that the child undergo genetic testing to confirm or rule out any genetic factors that may cause the motor deficit. A week later, the one-year-old child is taken in for genetic testing. A month later, the child is brought back to the pediatrician's office so that the pediatrician may discuss the genetic testing results with his parents. The genetic testing shows that the child has a mutation in the TAF1 gene and is diagnosed with TAF1 Intellectual Disability Syndrome (TAF1 ID syndrome). The pediatrician prescribes a composition comprising a therapeutic amount of SAK3 for the child to take orally. After six months of taking the SAK3 composition, the child shows improved motor skills and cognitive abilities. The pediatrician recommends continuing the use of the SAK3 composition for the time being. No side effects reported.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

1. A method of treating or improving motor deficits associated with TAF1 Intellectual Disability Syndrome (TAF1 ID) in a subject in need of such treatment, the method comprising administering a therapeutic amount of SAK3 to the subject.

2. The method of claim 1, wherein the subject for treatment is mammal.

3. The method of claim 1, wherein the subject for treatment is human.

4. The method of claim 1, wherein motor deficits refers to a partial or total loss of function of a body part.

5. The method of claim 1, wherein motor deficit refers to beam crossing time.

6. The method of claim 1, wherein the motor deficits refers to the number of foot slip errors.

7. The method of claim 1, wherein the method treats or improves neurological cognition.

8-14. (canceled)

15. A method of restoring protein levels and regenerating cells in vivo in a patient with TAF1 Intellectual Disability Syndrome (TAF1 ID), comprising administering SAK3 to the patient.

16. A method of claim 15, wherein the cells are Purkinje cells.

17. A method of claim 15, wherein the cells are astrocytes.

18. A method of claim 15, wherein the protein is Forkhead box protein P2 (FOXP2), or Brain-derived neurotrophic factor (BDNF).

19. A method of claim 15, wherein the protein is Brain-derived neurotrophic factor (BDNF).

20. A composition comprising a therapeutic amount of SAK3 for use in a method of treating TAF1 Intellectual Disability Syndrome (TAF1 ID), treating motor deficits associated with TAF1 ID, preventing motor deficits associated with TAF ID, or restoring protein levels and regenerating cells in vivo.

21-23. (canceled)

24. The composition of claim 20, wherein motor deficits refers to a partial or total loss of function of a body part.

25. The composition of claim 20, wherein the cells are astrocytes, Purkinje cells.

26. The composition of claim 20, wherein the protein is Forkhead box protein P2 (FOXP2).

27. The composition of claim 20, wherein the protein is Brain-derived neurotrophic factor (BDNF).
